(19) United States
(12) Patent Application Publication
(10) Pub. No.: US 2009/0173619 A1
(43) Pub. Date: Jul. 9, 2009

(54) APPARATUS AND METHOD FOR SELECTIVELY DISSOCIATING MOLECULES

(75) Inventor: Paul S. Banks, San Marcos, CA (US)

Correspondence Address: FITCH EVEN TABIN AND FLANNERY, 120 SOUTH LASALLE STREET, SUITE 1600, CHICAGO, IL, 60603-3406, US

(73) Assignee: GENERAL ATOMICS, A CALIFORNIA CORPORATION, San Diego, CA (US)

(21) Appl. No.: 11/971,894

(22) Filed: Jan. 9, 2008

(51) Int. Cl. B01J 19/08; C07C 1/00

(52) U.S. Cl. 204/157.15; 422/186

(57) ABSTRACT

An apparatus and method for selectively dissociating target molecular species present in a gas mixture is disclosed. An embodiment of an apparatus of the present invention comprises a containment structure for containing a gas mixture having a target molecular species; and a radiation source proximate to the containment structure and configured to apply electromagnetic energy to the gas mixture in the containment structure, the electromagnetic energy having a wavelength that dissociates molecules of the target species. An embodiment of a method of the present invention comprises the steps of containing a gas mixture having a heavy hydrocarbon; and applying to the gas mixture electromagnetic energy having a wavelength that dissociates molecules of the heavy hydrocarbon.

### BACKGROUND OF THE INVENTION

1. Field of the Invention

The present invention relates generally to selectively transforming large molecules to smaller ones. More specifically, the invention relates to a system and method for removing heavy hydrocarbons (tar) from gas mixtures such as synthesized gas (syngas).

2. Discussion of the Related Art

One source of renewable energy is living or recently living biological material such as wood, grass or other plant residues. The renewable biological material, referred to as biomass, can be used as a feedstock in the energy generation process. Energy produced from biomass is not only renewable but is also viewed as carbon neutral since biomass is part of the carbon cycle. This is because the carbons extracted from biomass in the energy generation process are eventually released into the atmosphere and converted back into other plant life through the process of photosynthesis.

To generate energy, biomass is gasified by heating a biomass feedstock to temperatures of over 700 degrees Celsius. At these elevated temperatures, the biomass reacts with oxygen creating a gas mixture known as synthesis gas (syngas). Syngas produced from the biomass is largely a mixture of carbon monoxide, hydrogen, carbon dioxide and water. Syngas is combustible and can be burned directly to produce energy. Syngas can also be further refined to produce synthetic gas and synthetic petroleum.

An unwanted product in the syngas generation process is heavy hydrocarbons (tar). These unwanted hydrocarbons usually represent less than 1% of the syngas mixture by molecular weight. The heavy hydrocarbons present in the syngas pose significant problems to turbines, other machinery, and subsequent catalysts and should be removed from the syngas mixture before the syngas is burned or refined.

There are a number of conventional techniques used to remove heavy hydrocarbons (tar) from syngas including thermal cracking, low temperature catalytic cracking, and scrubbing. Problems with each of these techniques make them commercially unviable. For example, thermal cracking requires heating the feedstock or syngas to temperatures of well over a 1000 degrees Celsius reducing the energy conversion efficiency. Catalytic cracking allows tar cracking at more energy efficient lower temperatures but requires the addition of costly catalysts during the tar removal process. Scrubbing results in tar being transferred to scrubbing liquids generating toxic or carcinogenic waste products or requiring costly recirculating systems.

The dissociation of molecules into their smaller components using optical radiation has long been practiced and is generally referred to as photodissociation. This term refers to the process whereby a photon with energy greater than the bandgap of the substance is absorbed by the electrons of the constituent atoms of the molecules. This causes the bond involving that electron to break, causing the dissociation of the molecule. It is even possible to select the bonds that will be broken in this fashion by carefully selectively the wavelength and bandwidth of the incident optical radiation. The atomic states accessed by this technique are typically high energy and require optical radiation in the ultraviolet or occasionally in the visible portions of the electromagnetic spectrum. Infrared photons do not have enough energy to access these atomic states, and cannot used for photodissociation as currently practiced. UV and visible lasers required for photodissociation are inefficient and expensive.

The rearrangement of hydrocarbon molecules by indirect heating of the surrounding gas through the application of microwave energy has been proposed. For example, in U.S. Pat. No. 3,663,394 Kawahara proposes using continuous wave microwave energy at 2.450 MHz, the resonant frequency of water, to rearrange carbon atoms having one to sixteen carbon atoms. Similarly, in U.S. Pat. No. 5,277,773, Murphy proposes converting C₁₊ hydrocarbons to unsaturated hydrocarbons and hydrogen by introducing water to the hydrocarbon feed stream and radiating with microwave energy at 2.450 MHz. In these methods, the microwave radiation interacting with the water in the gas serves only as a heating method, similar to the role of traditional combustion methods. In addition, this method heats the gas less efficiently than traditional methods because of the inefficiencies in generating the microwave radiation. The microwave radiation itself has no interaction directly with the hydrocarbons themselves. Rearranging hydrocarbon molecules as proposed by Kawahara and Murphy (i.e. through microwave heating of water molecules proximate to the hydrocarbon molecules) consumes a considerable amount of energy and therefore does not provide an energy efficient and economically sound process for removing heavy hydrocarbons from biomass syngas.

Those skilled in the art will recognize that there is a need for an energy efficient and economically viable apparatus and method for selectively dissociating molecules in gas mixtures. Applicant's invention addresses these needs as well as others.

### SUMMARY OF THE INVENTION

Several embodiments of the invention advantageously address the above stated needs as well as many others.

In one embodiment, the invention can be characterized as a method for selectively dissociating molecules of a substance, comprising applying electromagnetic radiation to the substance, the electromagnetic radiation being of a wavelength that lies within a vibrational or rotational absorption band of the molecules to be dissociated.

In another embodiment, the invention can be characterized as an apparatus for removing a heavy hydrocarbon from a synthesized gas mixture, the apparatus comprising: a first containment vessel where the synthesized gas mixture is formed or stored; a pipe having a window, the pipe connecting the first containment vessel with a second containment vessel and configured to allow synthesized gas to flow from the first containment vessel to the second containment vessel; and a radiation source optically coupled with the window of the pipe for radiating electromagnetic energy through the window, the electromagnetic energy having a wavelength that dissociates molecules of the heavy hydrocarbon.

In a further embodiment, the invention can be characterized as a method for removing hydrocarbons from a gas comprising: containing a gas mixture having a heavy hydrocarbon; and applying to the gas mixture, electromagnetic energy having a wavelength that dissociates molecules of the heavy hydrocarbon.

### BRIEF DESCRIPTION OF THE DRAWINGS

The above and other aspects, features and advantages of several embodiments of the present invention will be more apparent from the following more particular description thereof, presented in conjunction with the following drawings.

FIG. 1 shows a perspective drawing of a gas cleanup apparatus according to a first exemplary embodiment of the present invention;

FIG. 2 shows a perspective drawing of a second gas cleanup apparatus according to a second exemplary embodiment of the present invention;

FIG. 3 illustrates exemplary hydrogen carbon (H—C) bond absorption bands that may be exploited by the gas cleanup apparatuses of FIGS. 1 and 2 to selectively dissociate the H—C bond of heavy hydrocarbon molecules;

FIG. 4 is a flow diagram of a gas cleanup method according to an exemplary embodiment of the present invention;

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

The following description is not to be taken in a limiting sense, but is made merely for the purpose of describing the general principles of the invention. The scope of the invention should be determined with reference to the claims. The present embodiments address the problems described in the background while also addressing other additional problems as will be seen from the following detailed description.

Exemplary embodiments of the present invention are directed toward an apparatus and method that employ narrowband electromagnetic radiation to selectively dissociate selected molecules while in the presence of other molecules that are not affected (or are affected to a lesser extent) by the electromagnetic radiation. A preferred embodiment of the invention uses electromagnetic radiation with a wavelength that is resonantly absorbed by the vibrational and/or rotational structure of the selected molecules, rather than using electromagnetic radiation to generally excite electronic states of all of the molecules in the matter as is done in conventional molecular dissociation processes.

Selective dissociation of the molecules of heavy hydrocarbons through the application of electromagnetic energy at the vibrational and/or rotational resonance wavelength of the molecules is an energy efficient way to remove unwanted heavy hydrocarbons from matter. In particular, the application of the electromagnetic energy to biomass generated gas mixtures, such as syngas, provides an energy efficient process for removing the undesirable heavy hydrocarbons that usually represent less than 1% by of the molecular weight of syngas. This selective dissociation of heavy hydrocarbons using the apparatuses and methods disclosed herein contrasts with conventional techniques that inefficiently apply energy to the entire volume of syngas including its substantial water volume. The energy efficiencies achieved through selective dissociation of heavy hydrocarbons in syngas makes renewable biomass energy economically viable.

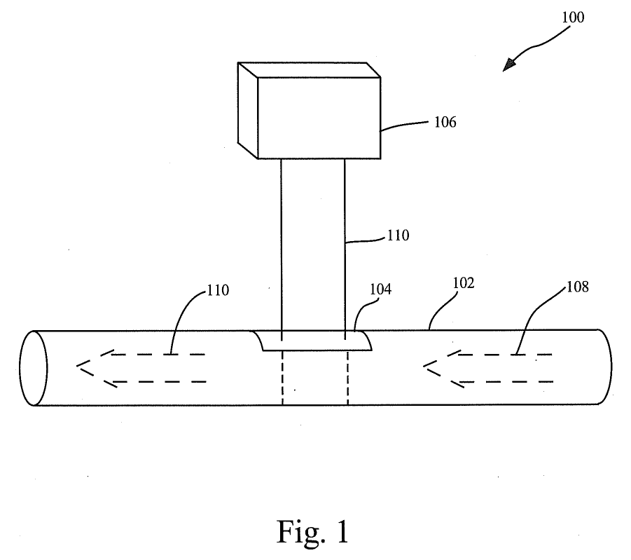

Referring to FIG. 1, FIG. 1 shows a perspective drawing of a first gas cleanup apparatus 100 according to a first exemplary embodiment of the present invention. The gas cleanup apparatus 100 includes a containment structure 102 having a window 104. The window is located proximate to a radiation source 106.

The containment structure 102 is configured to allow a gas mixture 108 having unwanted heavy hydrocarbon molecules to flow through the containment structure and near the window 104. The radiation source 106 generates electromagnetic energy 110 that propagates through the window 104 and interacts with the gas mixture 108 to produce a clean gas mixture 110 with a substantially reduced quantity of heavy hydrocarbon molecules.

The containment structure 102 can be any structure that contains the gas mixture. In this embodiment, the containment structure 102 is a cylindrical pipe having an inner diameter that is complementary to the flow rate of the gas mixture 108 and the energy content of the electromagnetic radiation 110. This allows energy efficient irradiation and dissociation of the heavy hydrocarbon molecules in the gas mixture 108 as they pass through the electromagnetic energy 110.

The window 104 provides a transparent medium for the electromagnetic energy 110 to enter the containment structure 102 while containing the gas mixture 108 inside the containment structure 102. In some embodiments, the window 104 may be heated to reduce gas mixture residues that may be deposited on the window 104. The window 104 in many embodiments is composed of optical material such as glass that allows visible or far infrared radiation to propagate through the window. Those skilled in the art will recognize that the optical materials chosen for the window 104 depend on the wavelength of the electromagnetic energy generated by the radiation source 106.

The radiation source 106 may be any source of electromagnetic radiation of sufficient spectral irradiance, including, for example, an LED, a laser diode, a laser, or other type of narrowband radiation source. The radiation source 106 preferably emits electromagnetic energy 110 at a wavelength in one or more of the rotational or vibrational absorption bands of the molecular bonds of a target molecule in the gas mixture (i.e. in an absorption band). For example, if the gas mixture is syngas the wavelength may be the resonant wavelength or overtone of a hydrogen-carbon bond in one of the heavy hydrocarbons in the syngas mixture (explained further hereinafter).

In this embodiment the gas mixture 108 is syngas produced through a biomass gasification process. The syngas mixture typically includes; hydrogen, carbon monoxide, carbon dioxide, water and heavy hydrocarbons. The heavy hydrocarbons in the syngas mixture are generally chains of hydrocarbons with relatively high condensation temperatures that make up approximately one percent of the syngas by molecular weight but can sometimes make up as much as ten percent or more of the syngas.

Advantageously, some of the absorption bands of hydrocarbons, such as those of the H—C bond, do not overlap the absorption bands of other molecules found in syngas, such as hydrogen, carbon monoxide, carbon dioxide and water. Because of this, electromagnetic radiation 110 applied to those particular hydrocarbon absorption bands is almost exclusively absorbed by the hydrocarbons in the gas mixture 108 and used to break the molecular bonds of the heavy hydrocarbons, resulting in the molecule breaking apart into smaller molecules. Consequently, very little energy is “wasted” stimulating other molecules in the mixture. This selective energy application is particularly important for mixtures such as syngas where the target molecules such as heavy hydrocarbon molecules generally represent a small fraction of the total molecules of the gas mixture 108.

Those skilled in the art will recognize that the apparatus described above may also be used for reducing heavy hydrocarbon content from non biomass generated syngas such as coal syngas as well. Moreover, the chemical dissociation process for heavy hydrocarbons is applicable to other chemical bonds and it is contemplated that the apparatus disclosed above may be use to transform or reduce other unwanted substances from gasses and liquids.

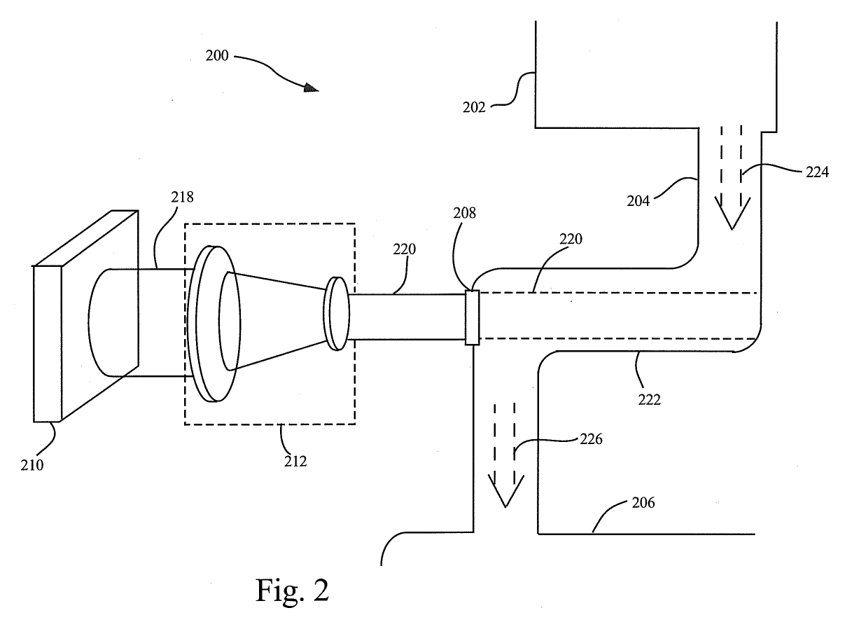

Referring next to FIG. 2, FIG. 2 shows a perspective drawing of a gas cleanup apparatus according to a second exemplary embodiment of the present invention.

A first containment vessel 202 is connected with a second containment vessel 206 through a pipe 204 that extends circuitously from the first containment vessel 202 to the second containment vessel 206. The pipe has a window 208. The window 208 is proximate to a radiation source 210. The window 208 and radiation source 210 may be separated by beam shaping module (BSM) 212 that may comprise one or more lenses.

The radiation source 210 emits electromagnetic radiation 218 that is shaped by the BSM 212 into a beam 220 having an envelope sized and shaped to enter the window 208 of the pipe 204. The pipe has a radiation segment 222 through which the beam 220 propagates. A gas mixture 224 flows from the first containment vessel 202 through the beam 220 that dissociates selected molecules (e.g. heavy hydrocarbons), as explained above, from the gas mixture 224 and a clean gas mixture 226 flows toward the second containment vessel 206.

Those skilled in the art will recognize that the flow rate of the gas mixture 224 or the output power of the radiation source 210 may be adjusted to control the amount of electromagnetic energy that is imparted to the selected molecules in the gas mixture 224. Similarly, the size of the pipe, the shape of the pipe, the direction of the flow as well as the size and power of the beam envelope may be adjusted to control the amount of electromagnetic energy imparted on the selected molecules. The mixture flow rate may also be controlled through pumps or natural diffusion.

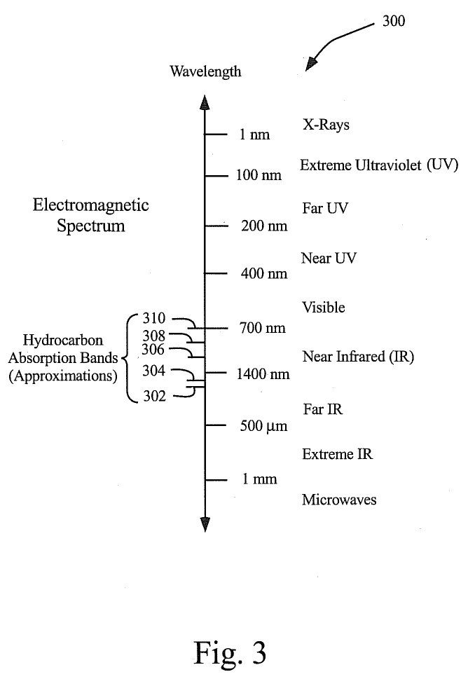

Referring next to FIG. 3, FIG. 3 illustrates exemplary hydrogen carbon (H—C) bond absorption bands that may be exploited by the gas cleanup apparatuses of FIGS. 1 and 2 to selectively dissociate heavy hydrocarbon molecules. An absorption band is a range of wavelengths within which electromagnetic energy is absorbed by a substance. Each substance has a characteristic set of absorption bands that are determined by the vibrational, rotational, electronic, and nuclear resonances present in the atoms and molecules that make up the substance. The vibration and rotational resonances of heavy hydrocarbons generally lay in the infrared and longer wavelength regions of the electromagnetic spectrum.

In the illustrated example, a first vibrational absorption band exists that includes a fundamental center wavelength 302 of approximately 3300 nm; a second vibrational absorption band exists that includes a first overtone 304 having a center wavelength of about 1650 nm; a third vibrational absorption band exists that includes a second overtone 306 having a center wavelength of about 1150 nm; a fourth vibrational absorption band exists that includes a third overtone 308 having a center wavelength of about 900 nm. A fifth vibrational absorption band exists that includes a fourth overtone 310 having a center wavelength of about 700 nm. For this example, the H—C bond absorption bands are generally about 100-200 nm wide, with the respective absorption bands centered near their respective fundamental or overtone center wavelength. Higher order overtones (not shown), as well as the fundamental and overtone wavelengths of the rotational resonance wavelength provide other heavy hydrocarbon absorption bands.

It can be recognized by those skilled in the art that the wavelengths referred to above at which the maximum absorption by a hydrocarbon compound of electromagnetic radiation having a wavelength within the respective H—C absorption bands 302, 304, 306, 308, 310 occurs, are approximations. It can also be recognized by those skilled in the art that the maximum absorption wavelength in a given hydrocarbon absorption band varies based at least upon the bond structure of the overall hydrocarbon compound. For example, CH₂, CH₃, CH₄ and a hydrocarbon having a ring structure may have varied absorption bands and overtone absorption bands including respective maximum absorption wavelengths that are different than the exemplary wavelengths.

Several embodiments of the invention, related to cleanup of heavy hydrocarbons (tars) in syngas, make use of the vibrational resonance of the hydrogen-carbon (H—C) bond that is at approximately 3300 nm or the first overtone at approximately 1650 nm. As explained above, the resonance wavelength varies slightly among the different hydrocarbon molecules because of the influence of the entire molecule on the exact resonance behavior. The H—C bond is particularly advantageous because the H—C bond is not present in the primary constituent gases (e.g. hydrogen, carbon dioxide, carbon monoxide, and water) present in syngas. In fact, the absorption cross section at 1700 nm(the peak of the first overtone absorption) in xylene, a typical tar component, is 2.4×10⁻²² cm² while that of water at the same wavelength is 0.17×10⁻²² cm². This makes it possible to absorb the dissociating radiation primarily in the small amount of hydrocarbon molecules.

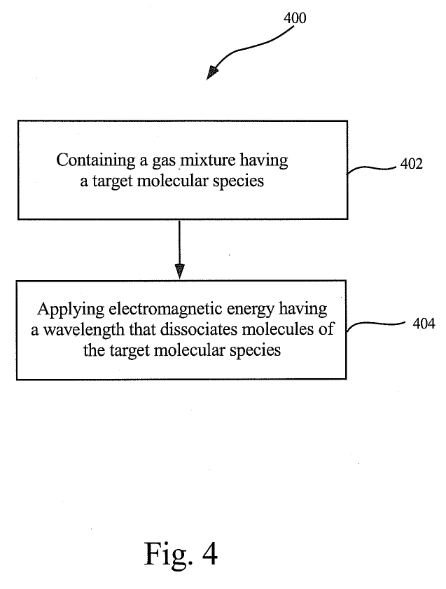

Referring to FIG. 4, FIG. 4 shows a flow diagram of a first exemplary gas cleanup method 400 according to an embodiment of the present invention. In a first step, a gas mixture having a target molecular species is contained (Step 402). The gas mixture may be, for example syngas, having an unwanted heavy hydrocarbon component. In a second step, electromagnetic energy having a wavelength that dissociates molecules of the target molecular species is applied to the gas mixture (step 404). The electromagnetic energy may have for example a wavelength that is the vibrational and/or rotational resonant wavelength of one of the molecular bonds of the heavy hydrocarbon as explained above. Alternatively, the electromagnetic energy may a wavelength that is an overtone of the vibrational or resonant wavelength of one of the molecular bonds of the heavy hydrocarbon.

The electromagnetic energy may be applied to the gas mixture by pumping the gas mixture through the electromagnetic energy. Alternatively the electromagnetic energy may be applied to the gas mixture by moving the radiation source or by moving lenses that propagate the electromagnetic energy toward the gas mixture.

While the invention herein disclosed has been described by means of specific embodiments and applications thereof, other modifications, variations, and arrangements of the present invention may be made in accordance with the above teachings other than as specifically described to practice the invention within the spirit and scope defined by the following claims.

What is claimed is:

1. A method for selectively dissociating molecules of a substance, comprising:
applying electromagnetic radiation to the substance, the electromagnetic radiation being of a wavelength that lies within a vibrational or rotational absorption band of the molecules.

2. The method of claim 1 wherein the selected molecules comprise a small percentage of a surrounding gas mixture.

3. The method of claim 1 wherein the wavelength of the applied electromagnetic radiation is in an infrared spectrum of approximately 800 nanometers to approximately 12,000 nanometers.

4. The method of claim 1 wherein the wavelength of the applied electromagnetic radiation is greater than 12,000 nanometers.

5. The method of claim 1 wherein the spectral irradiance of the electromagnetic radiation incident on the substance is greater than one watt/centimeter²/nanometer.

6. The method of claim 1 wherein the substance at least partially comprises carbon atoms.

7. The method of claim 1 further comprising, generating electromagnetic energy from a laser diode.

8. A method for selectively dissociating hydrocarbons in a mixture comprising:
applying electromagnetic radiation to the mixture including the hydrocarbons, the electromagnetic radiation being of a wavelength that lies within an vibrational or rotational absorption band of molecules of the hydrocarbon.

9. The method of claim 8 wherein the electromagnetic radiation has an optical wavelength and the optical wavelength is selected such that the electromagnetic radiation applied to the mixture is primarily absorbed by the molecules of the hydrocarbon.

10. The method of claim 8 wherein the optical radiation has an optical wavelength and the optical wavelength is substantially at or near a local absorption minimum of non-hydrocarbons substantively present in the mixture.

11. The method of claim 8 wherein the hydrocarbons comprise a small percentage of the mixture.

12. The method of claim 8 wherein the applied electromagnetic radiation has a wavelength that is in the infrared spectrum of approximately 800 nm to approximately 12,000 nm.

13. The method of claim 8 wherein the applied electromagnetic radiation has a wavelength that is greater than 12,000 nm.

14. The method of claim 8 wherein the spectral irradiance of the electromagnetic radiation incident on the substance is greater than one watt/centimeter²/nanometer.

15. The method of claim 8 further comprising the step of propagating the mixture toward a radiation source.

16. The method of claim 8 further comprising the step of propagating the mixture away from a radiation source.

17. The method of claim 8 wherein the mixture at least partially comprises hydrogen, carbon monoxide, carbon dioxide, and water.

18. The method of claim 8 wherein the hydrocarbons at least partially comprises molecules with six or more carbon atoms.

19. The method of claim 8 wherein the electromagnetic radiation is primarily absorbed by a hydrogen carbon bond of the hydrocarbons.

20. A hydrocarbon removal apparatus comprising:
a containment structure for containing a gas mixture having a heavy hydrocarbon; and
a radiation source proximate to the containment structure and configured to apply electromagnetic energy to the gas mixture within the containment structure, the electromagnetic energy having a wavelength at or near a vibrational or rotational resonance wavelength of the molecules of the heavy hydrocarbon.

21. The apparatus of claim 18 wherein the containment structure is for containing synthesized gas.

22. The apparatus of claim 18 wherein the containment structure has a pipe configured to allow syngas to flow toward the radiation source.

23. The apparatus of claim 18 wherein the containment structure has a pipe configured to allow syngas to flow away from the radiation source.

24. The apparatus of claim 18 wherein the radiation source is a laser diode.
